(19) United States
(12) Patent Application Publication
(10) Pub. No.: US 2007/0149886 A1
(43) Pub. Date: Jun. 28, 2007

(54) ECG recording device and method of use

(76) Inventor: Mark R. Kohls, New Berlin, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/319,640

(22) Filed: Dec. 28, 2005

(51) Int. Cl. A61B 5/04

(52) U.S. Cl. 600/509

(57) ABSTRACT

A portable, limited use electrocardiogram (ECG) recording device is provided for diagnosing cardiac problems. The device includes a cover hingedly connected to a base provided with a pair of spaced apart, tactile-sensing electrodes engageable with the patient's hands during an ECG recording and at least one indicator light signaling a start and finish status of the ECG recording. A circuit is electrically connected to the electrodes and the status indicator light and includes a processor and memory for receiving and storing ECG signals from the patient touching the electrodes. The cover has an outer face provided with preprinted mailing information for returning the recording device in a folded, closed position to a location for retroactively analyzing the stored ECG signals. The device may be disposed of following analysis of the stored ECG signals.

### FIELD OF THE INVENTION

This invention relates generally to the field of diagnostic cardiology, and more particularly, pertains to a portable, non-invasive recording apparatus capable of collecting information from a patient related to the patient's electrocardiogram (ECG) data at a location remote from a physician's office, clinic or hospital.

### BACKGROUND OF THE INVENTION

Syncope (sudden loss of consciousness) and arrhythmias (abnormal rhythm) resulting from electrical instability within the heart are particularly problematical for diagnostic physicians to observe in living patients. These events can be of short duration and sudden onset, and may occur very infrequently with little warning. Early diagnosis of arrhythmias is very important because the longer arrhythmia continues without detection or treatment, the greater the chances of severe permanent damage resulting in possible heart failure and stroke. Several arrhythmia-related diseases including Long QT Syndrome (LQTS), Wolff-Parkinson-White Syndrome, and Brugada Syndrome are genetic and LQTS can also be acquired through the use of certain pharmaceuticals. A clinically important method for diagnosing arrhythmia problems involves the use of electrocardiograms (ECGs) for monitoring the electrical activity in one's heart. Typically, electric leads are placed on the body at specific locations and the electrical activity resulting from heart depolarization and polarization is recorded by each lead. During a cardiac cycle, the electrocardiogram produces a distinctive waveform comprised of a P-wave, a QRS complex and a T-wave which are carefully analyzed to form diagnoses and assess the efficacy of treatments such as drug therapy.

ECG recorders are generally designed for portable collection of electrocardiograph data from a patient once an initial ECG is taken at a physician's office, clinic or other health facility having an on-site, non-ambulatory electrocardiograph. Recordings from these portable devices are subsequently used to detect abnormalities in the heart's electrical activity caused by a patient's routine daily activities or heightened emotional or physical states. Such recording devices are constructed of two types. The first is a retrospective type recording system that analyzes collected data after completion of the collection phase. The second type is a real-time system which analyzes data as it is recorded. In either type, the ECG signals are typically received through a plurality of leads attached between electrodes running between various points on a patient's body and an associated unit worn about the patient's neck, waist or wrist. The majority of these portable ambulatory monitors are bulky and interfere with the patient's normal life. Patient compliance cannot always be relied upon and is a common problem in their use. Problems with body-worn, lead-type portable monitors also include inability to abide attendant skin irritation. Removal may be required for showering, bathing or other water exposure. Results from such lead-type dependent monitors may vary depending on the placement of the various multiple electrodes upon the body of the patient.

Recently portable, non-ambulatory ECG monitoring and recording devices have become available which operate on tactile-sensing from thumbs or hands of patients without necessarily requiring leads and electrodes to be placed on the body. These recent portable, non-ambulatory devices are designed to be retained by the patient as long term, continuous use monitors/recorders which display real-time results and transfer collected data to another location. Such ECG result-displaying designs, while offering advantages over the prior art tend to be expensive to produce and maintain and, accordingly are not intended for large scale use. It is also important to consider a more basic type of portable device which does not require a display of real-time results, is more affordable, is designed for limited use, is returnable for analysis of the collected ECG data and is disposable.

One scenario which exemplifies the need for an improved, portable, non-ambulatory, tactile-sensing ECG monitor and recorder of the type that is inexpensive and readily in demand for a large group of patients relates to the dispensing of new pharmaceuticals. As is well known, new drugs follow a rigorous evaluation process from compound discovery to final approval requiring many years of trials and many millions of dollars in investment. Typically, a new drug is evaluated with progressive screening throughout Phase I, II and III clinical trials. Even after Phase III approval of the drug, liability concerns dictate a desire and/or requirement to perform a Phase IV trial, also known as post-market surveillance. These Phase IV trials are necessary because even if the drug showed few complications during Phase III, there is still no guarantee that when dispensed to a large number of patients that problems will not be discovered. A critical component of this drug follow-up is the recording of an ECG, primarily to look for the existence of drug-induced long QT syndrome (LQTS). Long QT syndrome causes an abnormality of the heart's electrical system predisposing certain affected persons to an extremely fast heart rhythm which can lead to sudden loss of consciousness and may cause sudden death. The name of the long QT syndrome refers to the QT interval measured on the ECG. To perform the test today requires a patient taking a medication to return to a physician's office, clinic or other facility with a substantially large, stand-alone electrocardiograph. This can be very time consuming as well as expensive and impractical to do with a drug monitored patient population that may be in the tens or even hundreds of thousands.

Besides LQTS, there is concern for monitoring congenital LQTS which is a genetic or inherited condition that can lead to fatal arrhythmia. These arrhythmias often occur in teenage children during physical exertion (e.g. playing a sport in school that involves swimming or running) and are frequently fatal. An improved portable ECG recorder is needed which would allow for a very inexpensive mass screening of athletes and other students to look for heart problems at an early age in a manner which is unavailable today.

Additionally, there exists today the problem in hospitals of infection control. Patients with severe infections, such as MRSA (methycillin-resistant Staphylococcus aureus), may require an ECG be acquired. Currently, a hospital electrocardiograph is used to make the recording. However, following the recording, the recording device must be disinfected before further use. This involves detailed cleaning using severe cleaning agents which can damage and eventually destroy the ECG leads. It is important to consider an alternative portable ECG design which will address the problem of infection control.

Accordingly, it is desirable to provide an improved easy-to-use, portable, non-ambulatory, tactile-sensing ECG recorder that can be produced inexpensively in large quantities, and is made available for large scale use outside a hospital, clinic or other ECG-equipped health facility. Such a portable ECG recorder should be capable of personal design for distribution to a patient picking up a particular prescription, as well as an anonymous design to be used for a generalized screening purpose. It is also desirable to provide a portable, lightweight ECG device which will read and store rather than actually display one or more ECG recordings, and which can be easily returned to a central lab for review and analysis. Ideally, such design is intended to have a limited use and be disposable once returned to a facility for interpretation of the collected ECG data. Additionally, the ECG recorder should be designed to be safely used in a high infection risk environment without the need to clean and disinfect.

### SUMMARY OF THE INVENTION

The present invention is directed to a portable limited use electrocardiogram (ECG) recording device used for diagnosing cardiac problems. The device is movable between a folded, closed position and an unfolded, ECG-recording operative position. The recording device includes a cover hingedly connected to a base. The cover and the base each have an inner face exposed to a patient when in the operative condition. The inner face of the cover is provided with operating instructions and a patient questionnaire. The inner face of the base is provided with a pair of spaced apart, tactile-sensing electrodes which are engageable with the patient's hands during an ECG recording. The inner face of the base further includes at least one indicator light for signaling a start and finish status of the ECG recording. A circuit is electrically connected to the electrodes and the status indicator light and includes a processor and memory for receiving and storing ECG signals from the patient touching the electrodes. The recorded ECG signals are sent to a remote location for retroactively analyzing the stored ECG signals.

In the preferred embodiment, the inner face of the cover includes pictorial operating instructions which are universally understandable to any patient. The inner face of the cover further includes a holder for receiving and retaining a first card having detailed text operating instructions printed in a desired language, and a second card for providing health and demographic information from the patient. The inner face of the base is provided with pictorial information reminding the patient to maintain their hands on the electrodes during the ECG recording, and to complete the patient questionnaire following completion of the ECG recording. The circuit is preferably formed as a card which is retained within the base. The circuit further includes a battery, leads connected to the electrodes, a user interface and an acquisition interface, all interconnected with the processor and the memory. The circuit card may include an internal connector adapted to be connected to a reading system for reading the stored ECG signal. The stored ECG signal is designed to be read such as by a wireless reading system, if desired. The base may also include an input connector in communication with the circuit and is adapted to receive at least one supplementary lead secured to an electrode positioned on a body portion of the patient. Additionally, the electrodes may also be used as a means of communication. The base may also have an outer face provided with an additional pair of spaced apart, tactile-sensing electrodes engageable with thighs of the patient. The inner face of the base may be formed with an indentation defining a sealable blood well for holding a blood sample of the patient. Preferably the recording device is provided along with medication prescribed to the patient. In this case, the circuit is preprogrammed according to the particular prescribed medication. The outer face of the cover also includes a bar code identifying the particular patient. The outer face of the cover further includes pre-paid postage and preprinted mailing information for returning the device.

The invention also contemplates a method of limited term monitoring of the patient for cardiac problems. The method includes the steps of providing a portable electrocardiogram (ECG) device which is movable between a folded, closed position and an unfolded, operative position wherein the device has a cover hingedly connected to a base, the base having at least one pair of spaced apart electrodes, at least one status indicator light and a powered circuit connected to the electrodes and the indicator light for recording and storing an ECG signal when the electrodes are touched; indicating a start status to the patient when the device is in the unfolded, operative position by means of the status indicator light to signify an ECG recording is required; touching the electrodes with hands of the patient; collecting and storing the ECG signal when the patient touches the electrode; indicating a finished status to the patient by means of the status indicator light to signify the ECG signal has been recorded; and transferring the stored ECG signal to a remote location for analysis thereof.

The method further includes disposing the device following analysis of the stored ECG signal. In the method, the device can be further provided with additional pair of spaced apart electrodes engageable with thighs of the patient, and the step of touching the electrodes includes touching with the thighs. The method also includes the step of notifying the patient with the results of the stored ECG signal following return of the device to the remote location.

In another aspect of the invention, there is provided a method of limited term monitoring of a patient for cardiac problems in conjunction with the taking of a particular prescribed medication. The method includes the steps of providing the patient with a portable personalized electrocardiogram (ECG) device at the time the prescribed medication is dispensed, the device being movable between a folded, closed position and an unfolded, operative position wherein the device has the cover hingedly connected to a base, the base having at least one pair of spaced apart electrodes, at least one status indicator light and a powered circuit connected to the electrodes and the indicator light for recording and storing and ECG signal when the electrodes are touched, the circuit being programmed in accordance with the particular prescribed medication; indicating a start status of the patient when the device is in the unfolded, operative position by means of the status indicator light to signify an ECG recording as required; touching the electrodes with hands of the patient; collecting and storing ECG signals when the patient touches the electrodes; indicating a finished status to the patient by means of the status indicator light to signify the ECG signal has been recorded; and transferring the stored ECG signal to a remote location for analysis thereof.

### BRIEF DESCRIPTION OF THE DRAWINGS

The drawings illustrate the best mode presently contemplated of carrying out the invention.

In the drawings:

FIG. 1 is a perspective view of an ECG recording device embodying the present invention, the device being shown in a folded, closed package;

FIG. 2 is a top view of the recording device of FIG. 1 in an unfolded, operative condition;

FIG. 3 is a bottom view of FIG. 2;

FIG. 4 is a view of a feedback card to be completed and returned by the patient after an ECG reading has been taken;

FIG. 5 is a view of a how-to-use card;

FIG. 6 is a block diagram of a circuit used in a recording device.

### DETAILED DESCRIPTION

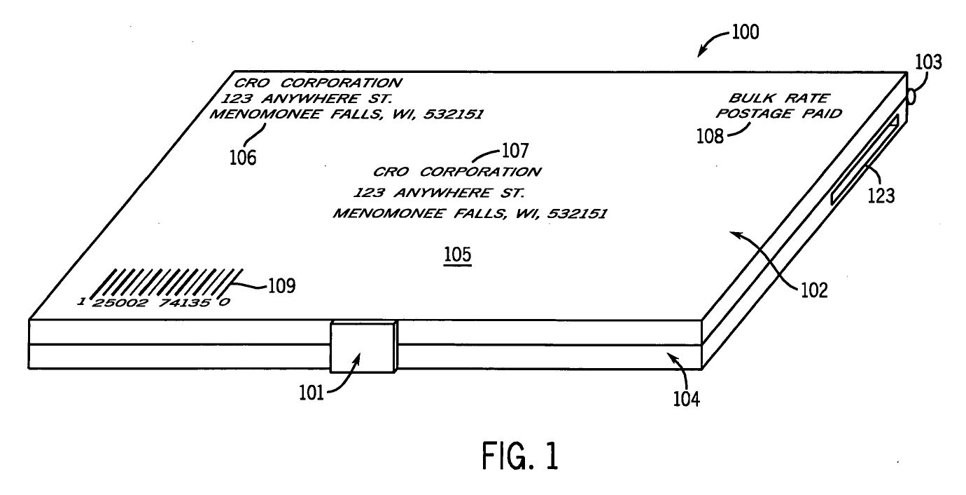

Referring now to the drawings, FIG. 1 illustrates a portable, non-ambulatory and non-invasive, electrocardiogram (ECG) recording device 100 used for monitoring and recording ECG waveforms by a patient preferably at a location remote from a physician's office, clinic or other ECG-equipped facility. The recording device 100 is dispensed, such as at a pharmacy or other outlet, in a package normally kept in a folded condition by any suitable closure member 101. The recording device 100 includes a planar cover 102 hingedly connected, such as by a spine 103, to a planar base 104. Both the cover 102 and the base 104 are constructed of a lightweight, rigid material, such as paperboard, foam or plastic, and are similarly sized and shaped. In the preferred embodiment, the cover 102 and the base 104 are both of rectangular shape with an exemplary width of 8 inches and a length of 11 inches. In the folded condition, the thickness of the package is typically less than one inch.

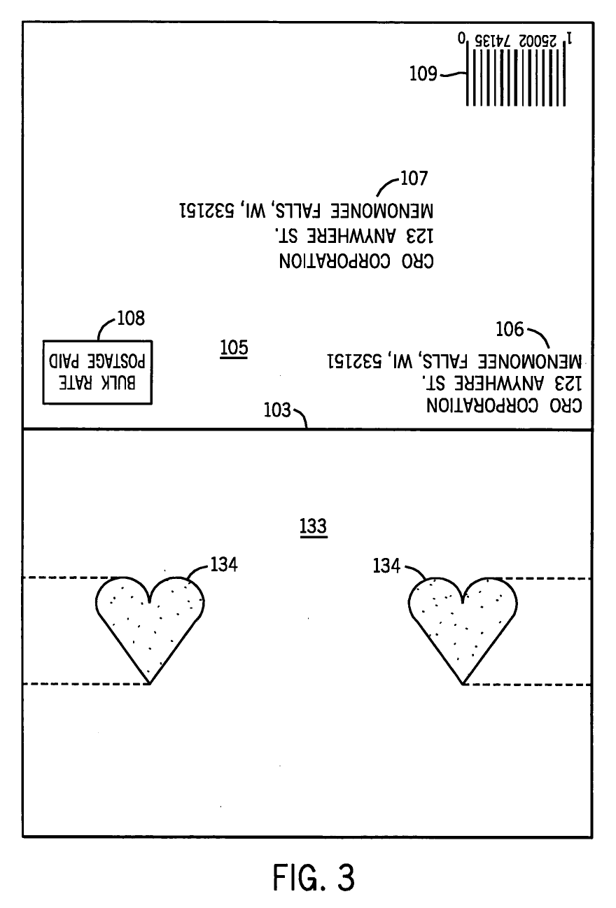

As will be further understood, the recording device 100 is designed as a self-mailer and the cover 102 has an outer face 105 provided with a return address 106, a mailing address 107, paid postage 108 and a bar code 109 which can be used to identify the patient receiving the device 100. FIG. 3 illustrates the outer face 105 of cover 102 in more detail, and shows a bottom view of the recording device 100 once closure 101 has been opened and the device 100 is in an unfolded operative condition.

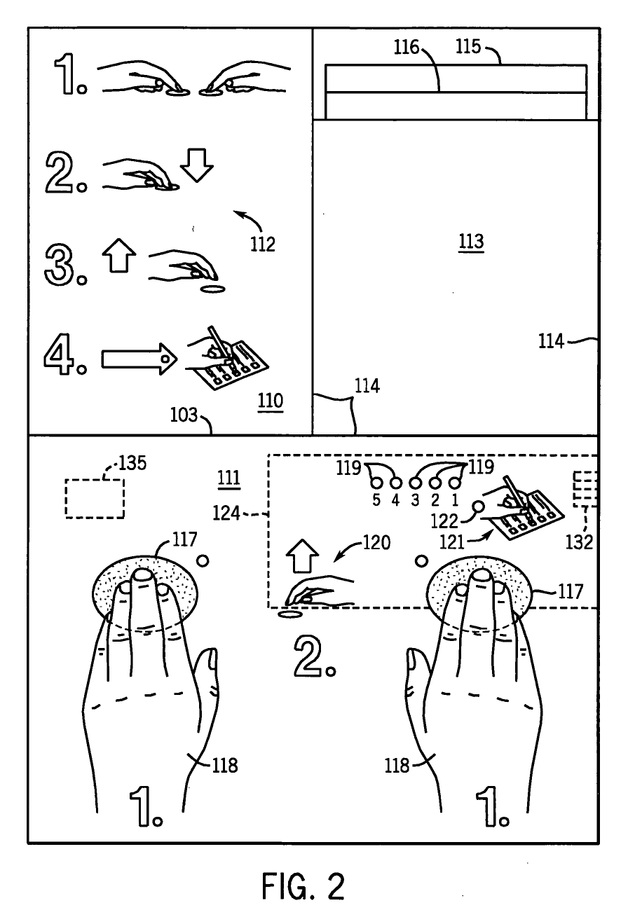

When the ECG recording device 100 is unfolded, as shown in FIG. 2, the patient views an inner face 110 of the folded back cover 102, and an inner face 111 of the base 104 which are divided by the spine 103.

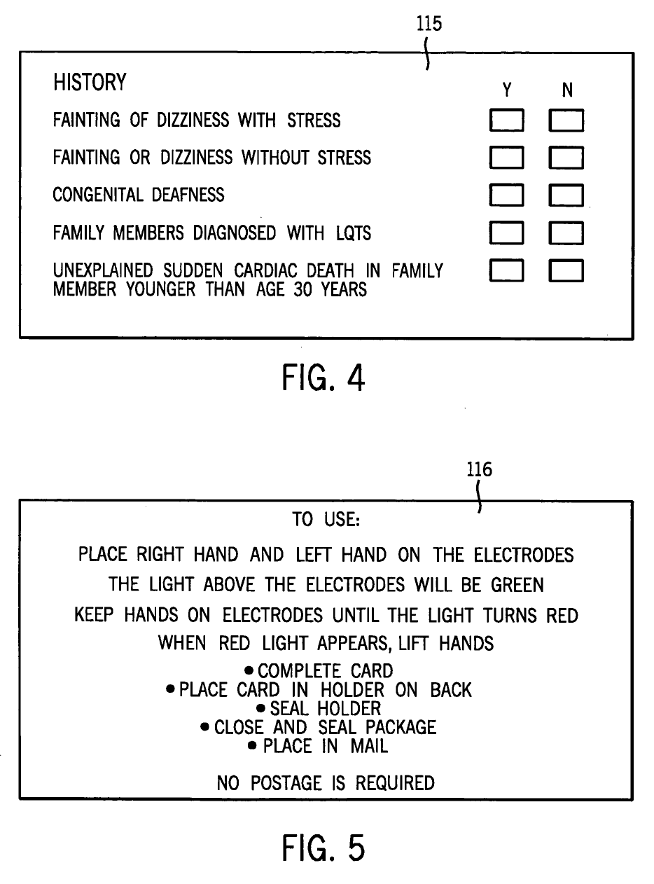

On one side, namely the left side, of inner face 110, a set of universally understandable pictorial instructions 112 is provided for guiding the patient in use of the device 100. On the opposite side, namely the right side of inner face 110 is a pocket or holder 113 generally defined by closed sides 114. The holder 113 is typically open at its top end to receive and retain a feedback card or questionnaire 115 and a more detailed instruction card 116 for operating device 100. The top end of holder 113 may be sealed. The feedback card 115 (FIG. 4) is particularly designed with a series of health and demographic questions for the patient to answer based on the medication prescribed to the patient with the dispensing of the recording device 100. The detailed instruction card 116 (FIG. 5) more precisely elaborates on the pictorial instructions 112, and may be written in a specific language for the particular patient.

The inner face 111 of base 104 is provided with a pair of spaced apart, tactile-sensing dry electrodes 117. The electrodes 117 are contacted by four fingers of the patient's right and left hands 118 during an ECG reading. At least one status indicator light 119 is included on the inner face 111 to provide a universally recognizable indication to the patient of the status of a particular ECG reading. In the example shown, there are several such indicator lights 119 to signify that multiple separate readings are required. In the preferred embodiment, each indicator light 119 changes color during a reading. For example, the indicator light 119 identified at “5” for the first of the readings, shows green indicating an ECG reading is ready to be taken. The patient's hands 118 are placed on the electrodes 117 and retained there until the light 119 turns to red indicating the termination of the first ECG reading. The lights 119 for the remaining ECG readings will operate identically and the patient will be instructed as to when to take the subsequent readings according to further information included on instruction card 116 and/or his/her particular instructions accompanying the drug prescription. A pictorial instruction 120 is provided on the inner face 111 between the electrodes 117 to remind the patient to hold their hands 118 upon the electrodes 117 until the particular indicator light 119 turns red at which time they may raise their hands 118 from the electrodes 117. A further pictorial instruction 121 on inner face 111 may include a reminder light 122 to instruct the patient to complete the questionnaire or feedback card 115 following the completion of an ECG reading.

As best seen in FIG. 1, the interior of base 104 is formed with a slot 123 for receiving and retaining a recoverable circuit card 124 which provides power, the necessary interconnections to the electrodes 117 and lights 119, and sufficient memory to record the ECG data. The circuit card is programmed in the preferred embodiment for the specific patient and his/her drug therapy. The circuit card 124 is removable from the device 100 after the device is returned by mail or similar carrier to a specific location to have the patient's ECG reading(s) analyzed.

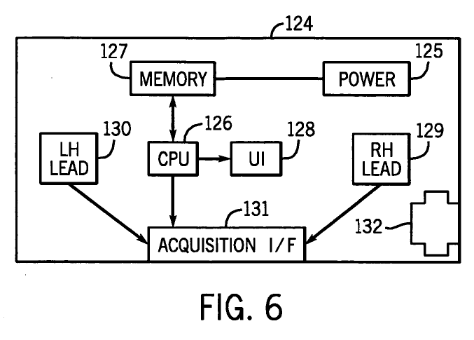

FIG. 6 shows a block diagram of the circuit card 124 which includes a source of power 125, such as a battery, interconnected to a central processing unit (CPU) 126 having a memory 127 in communication with a user interface (UI) 128. Right and left hand leads 129, 130 are joined to the electrodes 117. All electrical data sent from the patient through the electrodes 117 is converted to digital information stored in memory 127 by an acquisition interface 131 in electrical communication with elements 125-130. The circuit card 124 may also be optionally provided with an internal connector 132 where a reading system can be connected to the device 100 once the device is returned to its central location for analysis. This connector 132 can further be utilized in a mode to program the number of desired ECG readings or download additional information such as demographics or tracking information. Ideally, this connector 132 can implement a technology such as “near field communication” so that it would not be necessary to require actual physical connection to the device 100. Instead, the collected ECG data could be read out, such as by an IF or RF device. For infection control concerns, the device 100 with collected ECG data can be sealed in a sterile plastic bag and then safely read by scanning the protected device 100 using the IF or RF device.

In an enhanced version of the device 100, an outer face 133 of base 104 includes an additional pair of spaced apart, dry tactile-sensing electrodes 134 which are intended to contact the tops of the patient's right and left thighs and supply additional readings when the device 100 rests upon the patient's lap. The electrodes 134 may extend laterally to the side edges of base 104 (as shown in dotted lines) so that the device 100 may be alternatively placed between the patient's thighs. In this version, the electrodes 134 would have additional leads coupled to the circuit card 124.

Further optional considerations include the provision of a small indentation 135 for a sealable blood well along with a small pin (not shown) to draw blood from a finger. Such feature would allow for genetic testing which is useful in diagnosing arrhythmic abnormalities. In addition, a further input connector 136 may be provided within base 104 to provide a further connector point to circuit card 124 where a cable with additional disposable leads (not shown) as provided could be attached if desired for attachment to the patient's body. More leads would allow for the screening of more types arrhythmic conditions, such as Brugada syndrome. In a more advanced application, the device 100 could be preprogrammed to beep when it is time to take an ECG reading based on a protocol of a study. This would require insertion in the base 104 of a small audio device, such as a buzzer device, such as a buzzer or speaker.

The invention further contemplates transmission of the recorded ECG data back to the central receiving site without the need to mail back the device 100. Accordingly, further optional considerations include the provision of a speaker 137 on the base 104 that allows for an acoustically-coupled transmission. In this form, the user dials a phone number from his/her phone and places the phone mouthpiece over the speaker 137 so that recorded data is modulated at the normal voice frequency and decided at the central receiving site. In another version, it is envisioned adding a phone jack so that when the device 100 is plugged into the jack, it dials a preprogrammed phone number and enables a digital transmission back to the central site. Alternatively, a network connection can be added to permit a network transfer of ECG data over the Internet back to a preprogrammed central site. Furthermore, data could be optionally encrypted on the device 100 and during transmission of data.

In a preferred use of the basic device 100, a patient picking up a prescription, such as from a pharmacy, is concurrently giving the portable ECG recording device 100 which is preprogrammed to request a predetermined set of ECG readings at prescribed intervals based upon the patient's drug therapy. The device 100 is intended to be used by the patient at home or another location remote from a doctor's office, clinic or other facility with an electrocardiogram. Upon opening the device 100, the patient will remove and review the feedback and detailed use instruction cards 115, 116 from the holder 113. User instructions are pictorially summarized at 112 which are constantly visible to the patient. When ready to take an ECG, the patient positions the bottom of base 104 on his/her lap, and places the fingers of their hands 118 on the electrodes 117. The status indicator light(s) 119 will show green until the reading is finished and the light(s) will change to red. This prompts the patient to remove his/her hands 118 from the electrodes 117 and complete the feedback card 115. The completed card 115 is replaced in the holder 113. When any subsequent required recordings are completed, the entire recording device 100 is closed by closure member 101. The entire device 100 is then returned, such as by U.S. mail or the like, to a central location for analysis due to the self mailing design of the outer face 105 of cover 102. The patient can be automatically notified that the device 100 has been received. ECG data stored in the circuit card 124 is then read and analyzed, and the patient and the physician are advised of the results. Once the ECG data has been read, the entire device 100 is disposed of.

Alternatively, or in addition to, the system that reads the ECG data and programs the device 100 could also be programmed to call the patient on appropriate days to remind them to perform the ECG recording. In this option, the patient would need to register at a secure website. Registration at the website would also permit the patient to enter demographic and health information as well as a responses to the questions on the feedback card 115.

Device 100 having additional leads or other modifications would have an operational card 116 explaining their modifications.

While the ECG recording device 100 has been described as being used with prescribed medication, it should be understood that the device due to its affordability may be sold in retail stores to allow individual consumers to acquire ECGs that could be used to screen for other problems such as caused by allergies. The device 100 can be provided in schools and communities to enable screening, and may be useful in insurance evaluations and other applications.

The present invention thus provides a portable, non-ambulatory, non-invasive, disposable ECG recording device 100 which can be specifically designed to be given to and operated in a simple matter by a patient under a prescribed drug therapy. The device is designed to be produced in great quantities for a large scale use at a very low cost, is intended to read out data retrospectively, and can be branded or left anonymous. The packaging of the device is integrated for shipment back to a central location for analyzing the ECG recordings and reporting to the patient. The device can be used in a high risk environment safely without the need to clean and disinfect.

Various alternatives and embodiments are contemplated as being within the scope of the following claims particularly pointing out and distinctly claiming the subject matter regarded as the invention.

1. (canceled)

2. The device of claim 2, wherein the cover has an outer face configured to include mailing information for returning the recording device to the remote location for analyzing the ECG signals.

3. The device of claim 2, wherein the cover includes at least one of operating instructions and a patient questionnaire.

4. The device of claim 3, wherein the cover further includes a holder for receiving at least one of a first card having operating instructions and a second card for providing additional information from the patient.

5. The device of claim 2, wherein the device is provided with pictorial information reminding the patient to maintain the patient's hands on the electrodes during the ECG recording.

6. The device of claim 2, wherein the circuit is formed as a card retained inside the device.

7. The device of claim 2, wherein the circuit further includes a battery.

8. The device of claim 6, wherein the card includes an internal connector adapted to be connected to a reading system for reading the ECG signal.

9. The device of claim 2, wherein the ECG signals are read from the device by a wireless reading system.

10. The device of claim 2, further comprising an input connector in communication with the circuit and adapted to receive at least one lead secured to another electrode positionable on a body portion of the patient.

11. The device of claim 2, wherein the device comprises a second pair of spaced apart, tactile-sensing electrodes engageable with the patient during the ECG recording.

12. The device of claim 2, wherein the device includes an indentation defining a sealable blood well for holding a fluid sample from the patient.

13. (canceled)

14. The device of claim 2, wherein the circuit is preprogrammed to obtain a desired number of ECG recordings from the patient.

15. The device of claim 2, wherein the cover also includes patient identifying information.

16. The device of claim 2, wherein the outer face of the cover further includes prepaid postage.

17. (canceled)

18. (canceled)

19. (canceled)

20. (canceled)

21. The method of claim 31, wherein the step of transferring the device includes providing the device with mailing information for transferring the device to the remote location.

22. (canceled)

23. (canceled)

24. A portable, self-contained electrocardiogram (ECG) recording device, comprising:
a cover hingedly connected to a base, at least one of the cover and the base having a pair of spaced apart, tactile-sensing electrodes engageable with a patient during an ECG recording; and
a circuit electrically connected to the electrodes to the receive ECG signals from the patient touching the electrodes,
wherein the recording device is configured to be mailable to a location remote from a location of at least one of the ECG recordings.

25. The device of claim 24 further comprising a status indicator in communication with the circuit to provide feedback to the patient.

26. The device of claim 25 wherein the status indicator is one or more status indicator lights.

27. The device of claim 25 wherein the status indicator provides feedback to the patient when the device is in use.

28. The device of claim 11 wherein the second pair of tactile-sensing electrodes are engageable with either the patient's thighs or abdomen.

29. The device of claim 24 wherein the circuit includes a memory configured to store the ECG signals from the patient.

30. The device of claim 6 wherein the card is removable from the device.

31. A method of monitoring electrical activity of a patient's heart comprising:
a) providing a portable, self-contained electrocardiogram (ECG) device having a base including at least a first pair of spaced apart, tactile-sensing electrodes adapted to be touched by the patient and a powered circuit connected to the electrodes and operable for receiving an ECG signal from the patient when the electrodes are touched;
b) collecting the ECG signal when the patient touches the electrode; and
c) transferring the device to a remote location for analysis of the ECG signal.

32. The method of claim 31 further comprising the steps of:
indicating a start status to the patient to signify an ECG recording as required; and
indicating a finished status to the patient to signify the ECG signal has been collected.

33. The method of claim 31 further comprising the steps of:
receiving at least one or more fluids from the patient; and
storing the fluid within the ECG device.

34. The method of claim 31 further comprising the step of retrieving the stored ECG signals from the device using wireless communication.

35. The method of claim 31 wherein the ECG device includes a second pair of spaced apart, tactile-sensing electrodes engageable with the patient, wherein ECG signals are collected from both the first pair of electrodes and the second pair of electrodes.

36. The method of claim 33 wherein the second pair of electrodes are adapted to touch either the patient's thighs or abdomen.
